(19) United States
(12) Patent Application Publication
(10) Pub. No.: US 2025/0241772 A1
(43) Pub. Date: Jul. 31, 2025

(54) APPARATUS AND METHOD FOR TREATING VASOSPASM

(71) Applicant: PHENOX GMBH, Bochum (DE)

(72) Inventors: Hans HENKES, Stuttgart (DE); Hermann MONSTADT, Bochum (DE); Ralf HANNES, Dortmund (DE); Volker TRÖSKEN, Witten (DE)

(73) Assignee: PHENOX GMBH, Bochum (DE)

(21) Appl. No.: 18/997,086

(22) PCT Filed: Jul. 17, 2023
(86) PCT No.: PCT/IB2023/057287

§ 371 Date: Jan. 19, 2025

Related U.S. Application Data
(60) Provisional application No. 63/391,697, filed on Jul. 22, 2022.

(51) Int. Cl. A61F 2/88

(52) U.S. Cl. CPC A61F 2/88; A61F 2230/0023; A61F 2230/0026; A61F 2250/0036; A61F 2250/0059

(57) ABSTRACT

The invention relates to methods and corresponding device for treating a vasospasm with a stent structure, wherein the stent structure has an expanded state in which it lies against the inner wall of a blood vessel and a compressed state in which it is movable through the blood vessel within a catheter, wherein the stent structure is connected, preferably at its proximal end, to a delivery wire, wherein the stent structure is introduced within a catheter to the location in the blood vessel at which a vasospasm is present or predicted, the stent structure is released from the catheter, and the stent structure is moved longitudinally through the blood vessel in the expanded state. Optionally, the stent structure, after released from the catheter, the stent structure is left in position in the expanded state for a limited period of time followed by the longitudinal movement through the blood vessel.

### FIELD

The invention relates to a device comprising a stent structure intended for insertion into blood vessels of the human or animal body, and a method of using the stent structure to treat vasospasm.

### BACKGROUND

Vasospasm is a spasmodic constriction of a blood vessel. This is associated with the risk that subsequent vessels are no longer supplied with sufficient blood (ischemia), which can lead to necrosis of the tissue supplied with blood by the vessels. Especially in the cerebral area, vasospasm may occur several days after a subarachnoid hemorrhage (SAB), often as a result of rupture of an aneurysm. Other causes of subarachnoid hemorrhage include traumatic brain injury and bleeding from vascular malformations or tumors. Leaked blood in the subarachnoid space surrounds the vessels that run there and is considered the most important precipitating factor of vasospasm. Approximately 60% of all SAB patients experience vasospasm to a greater or lesser degree between the fifth and twentieth day after hemorrhage. When the arterial vessels are highly constricted, there is an undersupply of the dependent brain tissue, which can suffer irreversible damage as a result. Approximately 15-20% of primary SAB survivors experience permanent neurological damage with resulting disability. Approximately 5% of primary SAB survivors die later in life as a result of cerebral vasospasm. In this respect, vasospasm is one of the main reasons for strokes and even deaths occurring after rupture of an aneurysm and/or bleeding from the same or surgery in this area.

Usually, vasospasm is treated with drugs, especially calcium channel blockers or drugs that increase the level of NO in the blood. An example of a calcium channel blocker is nimodipine, which is often used after subarachnoid hemorrhage to prevent vasospasm. However, drug treatment is associated with not insignificant side effects and is also costly and time-consuming.

Other options for treating vasospasm include intensive medical measures such as raising arterial blood pressure and increasing circulating blood volume, dilating constricted vessels with the aid of a balloon, blocking the stellate ganglion, and surgically destroying sympathetic nerve fibers (sympathicolysis). These treatment methods are individually inconsistent in their efficacy, in part very costly, and often not sufficiently long lasting. Blockade of the stellate ganglion and surgical sympathicolysis are effective because the sympathetic nerve fibers in the wall of the cerebral arteries are significantly involved in the development of cerebral vasospasm. However, these procedures are inadequate for the full prevention and treatment of cerebral vasospasm because the blockade of the ganglion stellatum lasts only a few hours and surgical sympathectomy is limited to a narrowly circumscribed segment of the vessel that must be surgically dissected for this purpose.

Vascular endoprostheses, or stents, are often used to treat vascular stenosis and are permanently implanted at the site of vascular stenosis to keep the vessel open. Typically, stents have a tubular structure. Stents can be delivered to the target site through a catheter and expanded; in the case of self-expanding stents made of shape memory materials, this expansion and attachment to the inner vessel wall occurs autonomously. Alternatively, stents can be expanded using balloons onto which the stent is crimped or other mechanical methods. After final placement, only the stent itself remains at the target site; catheters, guidewires, and other devices are removed from the vasculature.

Basically, similar implants are also used to close aneurysms by placing them in front of the neck of an aneurysm. However, such flow diverters generally have a higher surface density than stents for the removal of stenoses. An example of a flow diverter is described in a PCT patent application publication No. WO 2008/107172A1. Even though a flow diverter is not used as a stent in the strict sense, a flow diverter can be described as a stent structure. In the same way, in the context of the present invention, structures of basically similar design are understood, regardless of whether they are actually used as a stent.

Stent structures are typically either laser-cut, resulting in a surface of struts with openings between them, or consist of a wire mesh. Other manufacturing techniques such as 3D printing are also conceivable.

WO 2017/207689 A1 discloses a stent structure used to treat a vasospasm. In particular, this has a uniform radial force over its entire effective length, i.e. the length over which the stent structure is in contact with the inner wall of the vessel. The stent structure is released from a catheter at the position of vasospasm and thereby expanded and reinserted into the catheter after a certain period of time, typically 1 to 10 min. Temporary expansion of the stent structure with a uniform radial force has been found to be an effective method for treating vasospasm. The aforementioned patent application also describes a method for determining radial forces, which is referred to in the context of the present invention.

Another device for the treatment of vasospasm can be found in WO 2018/046592 A1. The stent structure described here is characterized by electrical conductors via which pulses can be applied to the nerve fibers running in the vessel wall of the blood vessel, thereby preventing or treating a vasospasm.

Based on this prior art, the task was to provide a method as well as a device to further improve the treatment and prevention of vasospasm.

### BRIEF DESCRIPTION OF THE DRAWINGS

FIG. 1 is a perspective view of a first exemplary stent structure for treating vasospasm in accordance with the present teachings;

FIG. 1A is a close-up view of a radially outward portion of the first exemplary stent structure contacting the inner wall of the blood vessel in accordance with the present teachings;

FIGS. 1B-1G are perspective view of various cross-sectional configuration of the struts and/or wires of the stent structure in accordance with the present teachings;

FIG. 2 is a perspective view of a second exemplary stent structure for treating vasospasm in accordance with the present teachings;

FIG. 2A is a close-up view of a radially outward portion of the second exemplary stent structure contacting the inner wall of the blood vessel in accordance with the present teachings;

FIG. 3 is a perspective view of a successful vasospasm treatment with the stent structure according to the present teaching.

### DETAILED DESCRIPTION

The present invention relates to a method for treating a vasospasm with a stent structure. Said stent structure has an expanded state in which it lies against the inner wall of a blood vessel, and a compressed state in which it is movable through the blood vessel within a catheter. The stent structure being connected, preferably at its proximal end, to a delivery wire. The treatment method includes the following steps; (1) inserting a stent structure within a catheter to the treatment site in the blood vessel where vasospasm is present or threatened to occur, (2) deploying the stent structure at the treatment site, by releasing the stent structure from the catheter, (3) if necessary, leaving the stent structure in its radially expanded state in position for a limited period of time, and (4) moving the stent structure in the expanded state in the longitudinal direction through the blood vessel.

A vasospasm occurs as a sudden spasmodic constriction of a blood vessel as a result of a stimulus. It is a reaction of the body to (supposed) injury with the aim of limiting blood loss by constricting the blood vessel. Accordingly, it has previously been assumed that injury or irritation of the vessel wall should be avoided in the treatment of vasospasm.

The movement of the expanded stent structure in which stimulus the vessel wall in the area of the vasospasm has been found to be beneficial in the treatment of vasospasm. Such a stimulus is created by slowly moving the expanded stent structure longitudinally, causing friction between the stent structure and the vessel wall. It is believed that, to some extent, the movement of the stent structure also causes denudation of the tunica intima or intima, i.e., the inner layer of the blood vessel. This is a thin endothelial layer that provides a smooth inner surface of the blood vessel.

According to the present invention, at least partial denudation of the intima in the affected blood vessel by moving the expanded device in a longitudinal direction plays a role in the successful treatment of vasospasm. Specifically, according to one embodiment of the present teaching, the stent structure exerts a certain amount of radial force against the surrounding blood vessel wall as it moves longitudinally along the blood vessel. The radial force exerted on the vessel wall by a stent-like device is a function of compression relative to the uncompressed state. This means, the more the device is compressed, the more the radial force is exerted against its surrounding; likewise, the less the device is compressed, the less the radial force is exerted against its surrounding.

Unlike those device such as those described in WO 2017/207689 A1, with a primarily function of dilating, i.e., widening, the blood vessel, and thus would exerts a relatively greater radial force against the surrounding vessel wall in order to stabilize the device in treatment site, the stent structure in the present invention is intended to move longitudinally in the area of vasospasm. Hence, the radial force exerted against the surrounding vessel wall is generally lower than those device intended to be implanted without moving.

In addition to the above described radial force, the manner of how the radial force is applied by the device against the vessel wall is another important factor to the treatment of vasospasm. According to one embodiment of the present teaching, the cross-sectional configurations of the struts/wires also plays a role in the effectiveness of the treatment. The cross-sectional shape of the struts/wires could be of a general shape of a triangle or a partial triangle, a trapezoid, a blade, or a diamond. And, according to one embodiment of the present teaching, as the device expands radially, the edge of the struts/wire that is formed along a vertex of such cross-sectional shape contacts the surrounding vessel wall. Due to this specific configuration, a stent structure with such design exerts a higher radial force against the vessel wall than the stent structure made with round struts/wires.

According to another embodiment of the invention, the radial force exerted against the vessel wall is not only stationary, but is also dynamical, specifically in the manner of a longitudinal movement during the vasospasm treatment. According to one embodiment, the stent structure is configured to move in the longitudinal direction over a period of about 30-60 seconds, and over a length of about 5-50 mm. Typically, the longitudinal motion is induced by proximal withdrawn of the proximal end of the delivery wire by a clinician. According to one embodiment of the present teaching, a distal end of the delivery wire joins a proximal end of the stent structure. Such movement is in a manner of continuous and steady motion, where the speed is generally slower than retracting an implant back into its delivery catheter, for example, when retracting a thrombectomy instrument into the aspiration catheter.

According to one embodiment of the present invention, treatment of vasospasm includes at least two steps. First, the stent structure is deployed into its radially expanded profile at a treatment location, for example, where the vasospasm occurs or threatens to occur. And second, the stent structure is moved in the longitudinal direction in order to induce an additional stimulus on the vessel wall. In one embodiment, the deployed stent structure is positioned in the treatment location for a limited period of time after its deployment and before being subjected to longitudinal movement. In another embodiment, the stent structure is subjected to the longitudinal movement immediately after its deployment.

According to one embodiment of the present invention, the movement of the stent structure in the longitudinal direction is usually in a proximal direction. In other words, the stent structure is pulled proximally over the delivery wire. According to another embodiment of the present invention, the movement of the stent structure in the longitudinal direction is in a distal direction. That is, the stent structure is pushed distally over the delivery wire.

According to one embodiment of the present invention, the longitudinal movement of the stent structure occurs only once. According to another embodiment of the present invention, the longitudinal movement of the stent structure is repeated at least one time. According to yet another embodiment, the longitudinal movement of the stent structure is repeated several times.

According to one embodiment of the present invention, upon completion of the first longitudinal movement to induce an additional stimulus on the vessel wall, the stent structure is re-sheathed proximally into a catheter and released again at the treatment location, i.e. where the vasospasm occurs or threatens to occur, then followed by a second deployment and a second longitudinal movement. The described treatment can be repeated multiple times. In one embodiment, the longitudinal movement is repeated at least once on the same day. In another embodiment, such longitudinal movement is repeated at least once over several consecutive days. In yet another embodiment, such longitudinal movement is repeated after a few weeks, for example after 6, 12 and 24 weeks, to induce a lasting effect.

According to one embodiment of the present invention, a repetition of the procedure, i.e. longitudinal movement of the deployed stent structure, significantly improves the result vasospasm treatment. In some cases, two repetition of the procedure are sufficient. In one embodiment, the treatment procedure is conducted at the location where the vasospasm occurs or threatens to occur. In another embodiment, the above described treatment procedure is executed to the one branch of a bifurcation, in order to treat vasospasm of the adjacent vessel. For example, a treatment procedure in the area of the middle cerebral artery (MCA) in the sphenoidal segment Ml as well as in the distal internal carotid artery (ICA) also led to dilation of the proximal anterior cerebral artery (ACA). This means that when a vasospasm is treated with the above described method with the stent structure disclosed in the present invention, vasospasms in adjacent vessels or adjacent vessel sections are also released.

In some embodiment, upon treating vasospasm with above described methods, no lesion is left when an examination of the vessel walls performed a few days after the treatment using vessel wall imaging, for example, magnetic resonance angiography (MRA). Thus, the above described procedure according to the present invention affects the intima as desired, but not the media or the adventitia.

In some embodiment, additional drug treatment can be carried out along with the treatment using the device according to the present invention, for example, with nimodipine. In particular, this can be applied intra-arterially at the site of the vasospasm.

According to one embodiment of the present invention, the connection between the insertion wire and the stent structure could be non-detachable or detachable. The delivery wire is usually a wire of the type similarly used for implants. In the embodiment where the stent structure is intended to remain permanently in the vascular system, the delivery wire is detachably connected to the stent structure. According to one embodiment, the detachment mechanism between the delivery wire and the stent structure could be a mechanical, thermal or electrolytic detachment. Such a detachable connection to the stent structure is also possible according to the present invention as the stent structure is intended for temporary deployment at the site of the vasospasm. A stent structure with detachable connection to its delivery wire provides an option for the physician, depending on the situation, to release the stent structure inside the blood vessel, for example, if a retraction proves to be problematic, impracticable, or to induce a permanent radial force application. The delivery wire is preferably made of stainless steel, nitinol, or a cobalt-chromium alloy. According to the invention, a wire with an inner cavity or lumen is also considered to be a delivery wire.

According to one embodiment of the present invention, the delivery wire is preferably attached to the proximal end of the stent structure biased radially. In other words, the connection between the delivery wire and the stent structure is not located in the center of the stent structure, but eccentrically at or near the inner wall of the vessel. In this way, blood flow is impeded to be as little as possible. Furthermore, the eccentric arrangement of the delivery wire facilitates retraction of the stent structure into the catheter.

According to another embodiment of the present teaching, the delivery wire is connected to the stent structure at multiple places, including the proximal end of the stent structure. Multiple connections between the delivery wire and the stent structure provides, on one hand, a slightly stronger connection and, on the other hand, a greater obstruction of the blood flow, due to the additional struts or wires running in the center of the blood vessel. Thus, according to some embodiment, the proximal end of the stent structure, which tapers toward the delivery wire and therefore no longer fully abuts the inner wall of the vessel and can exert essentially no radial forces on it, can be kept shorter. In the case of a delivery wire that is connected to the stent structure at multiple points, the insertion wire generally extends more centrally.

The stent structure is delivered through a catheter, which may be a microcatheter, in particular. Especially when used in the neurovascular field, the use of a microcatheter is usually necessary. The stent structure can be advanced to the treatment location through the catheter. Alternatively, the stent structure is housed within a catheter, and the catheter is advanced to the treatment location together with the stent structure therein.

Often, before the stent structure is delivery to the treatment location by a catheter, a relatively large lumen guide catheter is first inserted, through which the small lumen catheter, then is advanced distally beyond a distal end of the guide catheter. For neurovascular applications, for example, a guide catheter is advanced from the groin to the carotid artery, followed by a catheter advancing through the guide catheter beyond the distal end of the guide catheter.

According to one embodiment of the present teaching, the deployment of the stent structure is accomplished by a relative movement between the catheter and the stent structure. In one embodiment, the stent structure is held in place and the catheter is withdrawn proximally until the stent structure is exposed and radially expands. In another embodiment, the stent structure is deployed by advancing distally relative to the catheter. In yet another embodiment, the stent structure is deployed by a combination of these movements.

According to one embodiment of the present teaching, the stent structure is configured to self-expanding radially upon being released from the catheter. For this purpose, a stent structure made of a material with shape memory properties, such as with the use of nickel-titanium alloys known under the name Nitinol; or superelastic property. One skilled in the art should recognize that other alloys or polymers with the ability to self-expand into a pre-determined shape can all be used for the purpose of the present invention. Thus, the exemplary embodiment disclosed herein should not be viewed as limiting.

According to one embodiment of the present invention, the stent structure could also be manufactured at least partially from a cobalt-chromium alloy, Cobalt-chromium-nickel alloys, and cobalt-chromium-nickel-molybdenum alloys. In one embodiment, these material is largely titanium-free, which further improves the properties. One example of the material choice is known under the name 35N LT®.

The alloys mentioned above in the context of the present invention are mere examples. It should be clear that the mention of metals as a component of this alloy does not exclude that the alloy contains further components. For example, a cobalt-chromium alloy may contain other constituents in addition to cobalt and chromium, such as nickel or molybdenum. Similarly, a platinum-iridium alloy need not have platinum and iridium as its sole components. It should also be clear that the exemplary alloy mentioned above could also include non-metals, such as carbon or nitrogen in addition to metals. The examples given includes certain superelastic/pseudoelastic alloys, X-ray visible alloys, cobalt-chromium alloys, etc. Other alloys suitable for the treatment method disclosed in the present invention should all be considered within the scope of the present invention.

According to another embodiment of the present teaching, the stent structure is built with DFT (drawn filled tubing) wires or struts. DFT wires have an interior of one metal and a sheath of another metal, so that the wire combines properties of both metals. In particular, DFT wires may have a radiopaque interior and a pseudoelastic sheath. Suitable materials include those mentioned above, in particular platinum alloys for inducing X-ray visibility and nickel-titanium alloys for pseudoelasticity.

Upon completing a treatment of the vasospasm, the stent structure is usually withdrawn back into the catheter. The catheter carrying the collapsed stent structure is then removed from the blood vessel According to one embodiment, the catheter is pushed distally over the steadily positioned stent structure, so that the stent structure collapses radially to assume its compressed state as the catheter slides over the entire length of the stent structure. According to another embodiment, the stent structure withdrawn proximally into the steadily positioned catheter. In yet another embodiment, a combination of the catheter distal movement and the stent structure proximal movements leads to the stent structure collapsing radially and re-sheathed inside the catheter. Once the stent structure is inside the catheter, a clinician can withdraw the entire system proximally outside of the blood vessel.

The stent structure is normally composed of interconnected struts or wires forming a mesh structure. In one embodiment, the stent structure made by laser cutting a tube with known technique to those skilled in the art. A laser-cut stent structure has a plurality of openings, or a mesh structure, distributed over the circumference of the stent structure. The advantage of a laser-cut stent structure is that nearly any form of the openings can be designed. One skilled in the art should understand that other forms of manufacturing processes can also be employed to make the stent structure, for example, galvanic or lithographic manufacturing, 3D printing or rapid prototyping. Thus, the exemplary embodiment as described above, should not be viewed as limiting.

In another embodiment, the stent structure can be made with braided wires. In this case, a plurality of wires typically run helically along the longitudinal axis with another plurality of wires running helically in the opposite directions. These have wires crossing over and under one another to form a honeycomb-shaped openings between the wires. The total number of wires used in making the stent structure could be anywhere from 8 to 64 and each wire could be a single wire or a strand of wire (several wires of small diameter twisted together forming a filament). The advantage of a braided stent structure is that the wires can have any pre-shaped cross sections such as a triangle, trapezoid, blade-shape or a diamond-shape.

In one embodiment, the stent structure has interconnected struts such as a laser cut stent structure. Such stent structure tend to contract less in length during expansion. A stent structure of interconnected struts exert a relatively greater radial force than a braided stent structure, given the otherwise comparable construction, such as strut/wire density and strut/wire thickness. This is because the interconnected struts have a fixed connection at the intersections whereas the wires of a braided stent structure generally sliding over each other during radial expansion of the stent structure.

The present invention further provides an exemplarity struts/wire configuration for the purpose of intensify the treatment of the vessel wall in the area of the vasospasm. Specifically, according one embodiment of the present teaching, the cross-section of the struts/wires formed the stent structure, viewed radially, decreases in a radially outward direction. Upon deployment of the stent structure, the stent structure expands radially with the most radially outward portion of the struts/wires being in contact with the surrounding vessel wall. In one embodiment, the portion of the struts/wires contacting the blood vessel wall is relatively narrower than the radially inward portions of the struts/wires that do not contact vessel wall, i.e. the cross section of the struts/wires taper toward the radially outside direction, forming a reduced cross-sectional area. Such design of the struts/wires result in a stronger punctual pressure on the vessel wall, thus a stronger stimuli to the vessel wall with the same radial exertion force of the stent structure. According to one embodiment of the present teaching, the struts/wires with a cross-sectional shape of a triangle, wedge, trapezoid, blade or a diamond, exert a higher punctual force on the vessel wall than the stent structure made with struts/wires with a round cross-sectional shape.

Appropriately configured struts or wires can also provide some ablation of the intimal layer (denudation). By tapering the struts/wires outwards radially, the struts/wires can have a blade-like effect, and therefore enhances the denudation. This requirements are somewhat opposite to those for thrombectomy, i.e., removal of thrombi. In thrombectomy, it is desirable for the stent structure to be atraumatic, whereas in vasospasm treatment, according to the present invention, the blood vessel wall is to be influenced to a certain degree. Accordingly, the specific configuration of the struts/wires as disclosed herein is advantageous.

According to one embodiment of the present teaching, the struts/wires can, for example, have a triangular, wedge, trapezoidal, blade or a diamond-shaped cross-section. When the stent structure are made with such struts/wire fully expanded, the struts/wires are oriented in such a way that, in a cross-sectional view of the stent structure, the corner of the shaped wire is at the most radially outward portion of the stent structure and contacts the blood vessel wall.

According to another embodiment of the present teaching, at least one strut/wire of the stent structure has a quadrilateral cross-sectional shape which may include a parallelogram, a rectangle, a diamond, or any modified quadrilateral shape such as a rectangle with one inclined side. Considering one possible embodiment, when viewed in the cross-sectional direction, one of the acute angle of the quadrilateral is at the most radially outward part of the deployed stent structure. In other words, the outermost edge of the strut/wire formed along this corner is the most radially outward portion of the stent structure and contacts the blood vessel wall.

In the embodiment where the struts or wires extends diagonally, that is not oriented at right angles to the longitudinal axis, the edge of the struts/wire at the most radially outward portion of the stent structure is oriented toward a proximal direction. In this design, when the stent structure moves in the proximal direction, the strut/wire is oriented in a way that acts on the vessel wall in a similar way to a scraper or razor blade that could cause denudation there.

According to another embodiment of the present teaching, in order to produce a sufficiently strong effect on the vessel wall, the stent structure is configured so that when the stent structure is in its radially expanded deployed configuration, the struts/wires are oriented in a generally orthogonal direction to the longitudinal axis of the stent structure. For example, the struts/wires and the longitudinal axis of the stent structure form an angle of at least 60°. In one preferred embodiment, the angle between the struts/wire and the longitudinal axis of the stent structure is greater than 70°. In another embodiment, the angle between the struts/wire and the longitudinal axis of the stent structure is greater than 80°. Accordingly, when the stent structure moves in the longitudinal direction, a larger area of the vessel wall is affected.

The terms “proximal” and “distal” should be understood to mean that, when the device is inserted, portions of the device that are closer to the treating physician are referred to as proximal, and portions away from the treating physician are referred to as distal. Thus, the device is typically advanced through a catheter in a distal direction. The term “axial” refers to the longitudinal axis of the device running from proximal end to distal end, and the term “radial” refers to planes perpendicular the longitudinal axis.

In one embodiment, the openings formed in the stent structure between the individual struts/wires have an inscribed diameter of 0.1 to 6 mm, where an inscribed diameter is understood to be the diameter of the largest possible circle that can be placed in the opening. The maximum inscribed diameter refers to the stent structure in the fully unconstrained state, i.e. maximum radial expanded state. But depending on the diameter of the blood vessels in which the implant is placed, the implant may not be able to assume its fully expanded state, causing the inscribed diameter to differ from the maximal expanded state.

According to one embodiment, the stent structure has openings, formed between the individual struts/wires, with an inscribed diameter of greater 1 mm. Such relatively coarse-mesh stent structure is configured to exert a radial force of an appropriate magnitude to treat vasospasm. For example, a stent structure with an expanded diameter of 3 to 5 mm may have mesh openings with inscribed diameter of 2 to 4.5 mm.

According to one embodiment, the stent structure of the present invention has close cell mesh openings, i.e. each cell opening is a closed shape fully surrounded by struts/wires without interruptions.

In one embodiment, the stent structure of the present invention is constructed with struts/wires which have a relatively large cross-sectional area or diameter, i.e. relatively solid struts/wires, which serve the purpose of generating a specified magnitude of radial force. In one embodiment, in which the cross section may be round or angled, a height and a width of the struts/wires is of 30 to 300 μm, preferably of 40 to 200 μm, and most preferably of 40 to 120 μm, in order to generate a specific amount of radial force.

According to one embodiment of the present teaching, the stent structure has a generally longitudinal tubular opening extending from an open proximal end to an open distal end. The stent structure of such design has the advantage of interfering blood flow as minimally as possible, and preventing an undersupply of blood to the surrounding tissue. In another embodiment of the present teaching, the stent structure has a generally longitudinal tubular opening extended from an open proximal end to a closed distal end. The stent structure with closed distal end is more atraumatic. In this context, an open end means that there are no struts/wires at the respective end of the stent structure and that struts/wires are limited to the outer circumference of the stent structure. A closed end, on the other hand, is defined by the presence struts/wires in the center of the longitudinal tubular opening of the stent structure. Since the stent structure has mesh openings between the struts/wires along its outer circumference, even with a closed distal end, blood can still pass through the stent structure through its mesh openings, and thus, blood flow would be limited but not completely blocked.

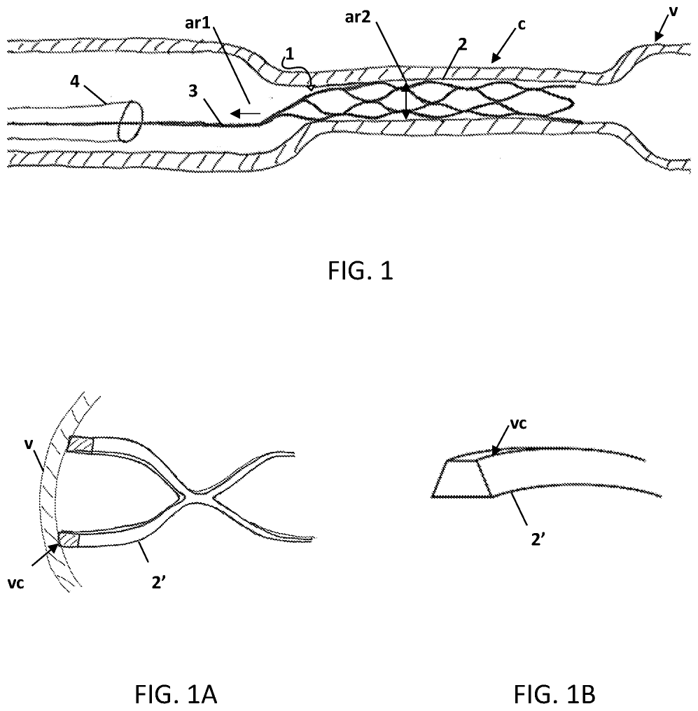

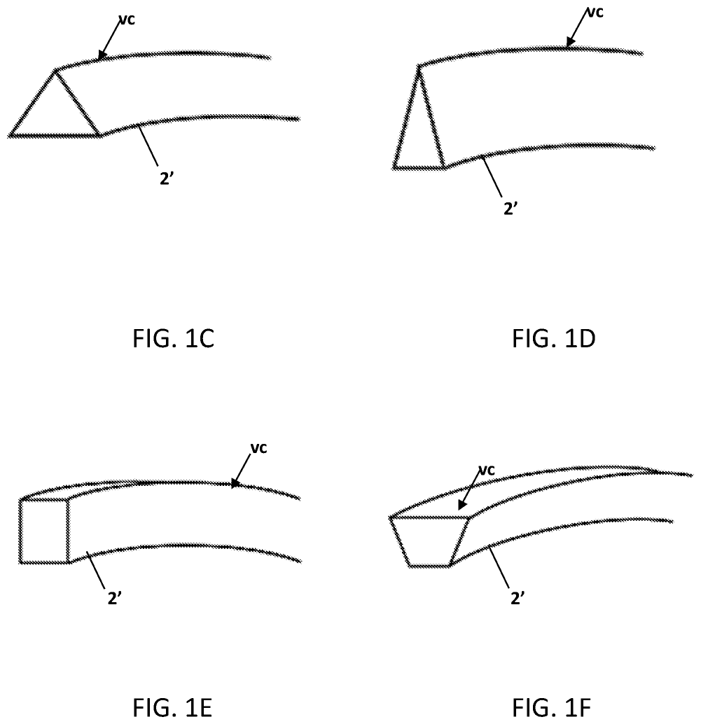

FIGS. 1-3 provide exemplary embodiments of the present invention. In all figures proximal is to the left and distal is to the right.

FIG. 1 shows a first exemplary embodiment of the present invention, a device for treating a vasospasm. The device 1 comprises a laser cut stent structure 2, which is connected at its proximal end to a delivery wire 3. The stent structure 2 is shown in its radially expanded state, outside the catheter 4 and inside the blood vessel area c (constricted area). According to one embodiment, the deployed stent structure 2 is positioned against the inner wall of the blood vessel constricted by a vasospasm. According to one embodiment, the stent structure 2 is delivered through a catheter (not shown). During the delivery, the stent structure 2 is compressed radially and placed inside a distal portion of the catheter 4. The catheter 4, carrying the stent structure 2, is inserted through the blood vessel v reaching the vasospasm site c.

FIG. 1 further shows a device and a method of treating a vasospasm with the inventive device 1 according to one embodiment of the present teaching. As the stent structure 2 is released from the catheter 4. It expands radially and abuts the inner wall of a blood vessel v at vasospasm site c. The stent structure is then placed at the vasospasm site c for a limited period of time followed by a movement of the stent structure 2 in its radially expanded state in the longitudinal direction through the constricted region c of the blood vessel v. Preferably, the movement of the radially expanded stent structure 2 is in the proximal longitudinal direction as indicated by first arrows ar1. In this context, the combination of the longitudinal movement and radial force of the device exerted onto the vessel wall in the constricted area c as indicated by second arrows ar2 results at least to a partial denudation of the inner layer of the blood vessel in the constricted area c, namely of the tunica intima or intima.

FIG. 1A illustrates a detailed, cross-sectional view of the stent structuring, with a most radially outward portion of the stent structure contacting the blood vessel wall. As can be seen from FIG. 1A, the radially outer surface of the stent struts 2′ is in contact with the vessel wall v.

FIG. 1B to FIG. 1G illustrates various embodiment of the cross-sectional configurations of the stent struts 2′. In one embodiment, as shown in FIG. 1 C, the cross-sectional configuration of the struts 2′ has at least partially a trapezoidal shape. In one embodiment, the shorter parallel sides is at the radially outside surface of the stent structure, and in contact with the vessel wall when the stent structure is in its radially expanded state. In another embodiment, a corner of the shorter parallel sides is at the most radially outward edge of the stent structure, which is in contact with the vessel wall when the stent structure is in its radially expanded state.

In another embodiment as shown in FIG. 1D, the cross-sectional configuration of the struts 2′ has at least partially a triangular shape. In one embodiment, one corner of the triangle is at the most radially outward edge of the stent structure, which is in contact with the vessel wall when the stent structure is in its radially expanded state.

In another embodiment as shown in FIG. 1E, the cross-sectional configuration of the struts 2′ has at least partially a wedge shape, i.e. an acute triangular shape. In one embodiment, one corner of the triangle is at the most radially outward edge of the stent structure, which is in contact with the vessel wall when the stent structure is in its radially expanded state.

In another embodiment as shown in FIG. 1F, the cross-sectional configuration of the struts 2′ has at least partially a parallelogram shape. In one embodiment, one side of the parallelogram is at the radially outside surface of the stent structure, and in contact with the vessel wall when the stent structure is in its radially expanded state. In another embodiment, a corner of the parallelogram is at the most radially outward edge of the stent structure, which is in contact with the vessel wall when the stent structure is in its radially expanded state.

In yet another embodiment as shown in FIG. 1G, the cross-section configuration of the struts 2′ has at least partially a rectangular shape. In one embodiment, one side of the rectangle is at the radial outside surface of the stent structure, and in contact with the vessel wall when the stent structure is in its radially expanded state. In another embodiment, a corner of the rectangle is at the most radially outward edge of the stent structure, which is in contact with the vessel wall when the stent structure is in its radially expanded state. In yet another embodiment, the corner of the rectangle forming the most radially outward edge of the stent structure is an acute angle.

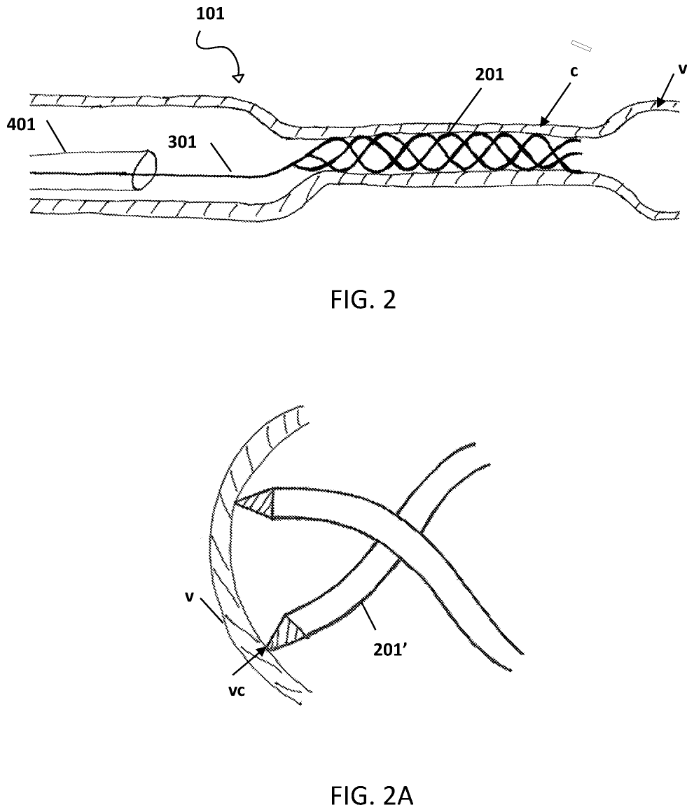

One skilled in the art should understand that the term “struts” used herein can also be wires, and these two terms may be used interchangeably. Thus, the various embodiments described with reference to FIGS. 1B-1G is also applicable to wires, for example the various embodiment described with reference to FIG. 2 and FIG. 2A.

FIG. 2 shows a second exemplary embodiment of the present invention, a device 101 for treating vasospasm. The device 101 comprises a braided stent structure 201 made of wires that is connected at its proximal end to a delivery wire 301. The stent structure 201 is shown in its radially expanded state, outside the catheter 401 and inside the blood vessel area c (constricted are). According to one embodiment, the deployed stent structure 2 is positioned against the inner wall of the blood vessel constricted by a vasospasm. According to one embodiment, the stent structure 201 is delivered through a catheter (not shown). During delivery, the stent structure 201 is compressed radially and placed inside a distal portion of the catheter 401. The catheter 401, carrying the stent structure 201, is inserted through the blood vessel v reaching the vasospasm site c.

FIG. 2A illustrates a perspective cross-sectional configuration through the constricted area c according to FIG. 2. As can be seen from FIG. 2A, the radial outer surface of the wires 201′ is in contact with the vessel wall vc.

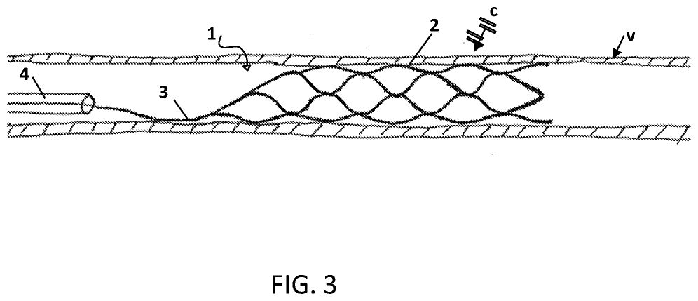

FIG. 3 illustrates the blood vessel v that was successfully treated with the device 1, according to one embodiment of the present teaching. As shown in the figure, The previously constricted area c is fully dilated, and the stent structures 2 expands radially further comparing to its expanded states as shown in FIG. 1. The stent structure 2 can now be retracted back into the catheter 4 and removed from the vessel v.

In one embodiment of the present teaching, an anti-thrombogenic coating on the inside of the stent structure is useful because it remains in the blood vessel for a certain time, during which time the formation of a thrombus in the blood vessel, which is already constricted by the occurrence of vasospasm, must be avoided. On the outside of the stent structure, a vasorelaxant coating is advantageous, for example, with a calcium channel blocker such as nimodipine.

In another embodiment, a coating that prevents adhesion and aggregation of platelets can also be incorporated to the stent structure, such as a coating as described in WO 2018/210989 A1. Here, a coating with a functional layer is applied to the medical device, wherein the functional layer comprises at least one sugar alcohol and/or is formed by an oligo- or polymerization of monosaccharides functionalized with polymerizable groups. This coating is capable of mimicking the natural glycocalyx.

Even though an antithrombogenic coating is particularly useful on the inner side of the stent structure and a vascular relaxant coating is particularly useful on the outer side of the stent structure, the antithrombogenic or vascular relaxant coating can also be applied to the stent structure as a whole, or the struts/wires can have a corresponding coating on all sides. In this case, the coating is not limited to the inside or outside of the stent structure.

In general, it should be noted that all coatings could be incorporated into a part or a partial length of the stent structure. In one embodiment, a coating is incorporated into the areas of the stent structure that contact the ‘inner wall of the vessel, i.e. the cylindrical part of the stent structure.

In one embodiment, the force exerted outward radially onto the inner wall of the blood vessel by the expanded stent structure should be between 2 and 30 N/m, preferably between 5 and 10 N/m, based on the stent structure with a general diameter of 2.00 mm. The specification of the radial force refers to the force exerted radially per unit length, i.e. it is the relative radial force. Only that part of the stent structure is taken into account which is in contact with the inner wall of the vessel and is therefore capable of exerting forces on it (effective length).

According to one embodiment of the present teaching, the radial force exerted by the stent structure in the expanded state is essentially constant along its effective working length, i.e. the radial force on both proximal section, middle section, and distal sections of the effective working length stent structure are the same. In another embodiment, a stent structure could exert weaker radial force at its proximal and distal sections of the effective working length, and a relatively stronger radial force in its middle section of the effective working length. In one embodiment, the proximal and distal sections of the stent structure are selectively modified in order to create a uniform radial force throughout the entire effective working length of the stent structure from its proximal end to its distal end. The proximal end of the effective working length of the stent structure is different from the proximal end of the stent structure. The proximal end of the stent structure refers to the most proximal portion of the stent structure that is no longer part of the effective working length, and where the struts/wires converge toward the delivery wire. According to one embodiment of the present teaching, a typical length between the proximal end of the stent structure and the proximal end of the effective working length of the stent structure is about 8 to 10 mm.

According to one embodiment of the present teaching, to increase the radial force in the proximal and distal sections of the effective working length of the stent structure, the struts/wires used in these two sections may have a greater cross-sectional area than those used in the middle section. In another words, the struts/wires used in these two section are greater in mass. Doing so, the basic tendency of a stent structure where the middle section exerts higher radial forces could be at least partially compensated.

In an alternative embodiment, struts/wires use to for proximal and distal sections of the effective working length of the stent structure could have a higher density than those used in the middle section of the working length of the stent structure. As such, the natural tendency of weaker radial force exerted by the proximal and distal sections of the stent structure is at least partially compensated.

According to another embodiment of the present teaching, it is possible to provide the stent structure with a slot that extends helically over the lateral surface of the stent structure, or longitudinally along the lateral surface of the stent structure. In some embodiment, at least one strut/wire can be incorporated across the slot, in order to adjust the radial force distribution longitudinally along the effective working length of the stent structure when the stent structure is in its radially expanded configuration.

In one embodiment, the diameter of the stent structure in its radially expanded state is typically in the range of 2 to 8 mm, preferably in the range of 4 to 6 mm. In another embodiment, the overall length of the stent structure in its radially expanded state is typically 5 to 50 mm, preferably 10 to 45 mm, more preferably 20 to 40 mm. In yet another embodiment, the effective length, i.e., the length of the stent structure in the radially expanded state that actually exerts radial forces on the inner vessel wall, is usually about 8 to 10 mm shorter than the overall length of the stent structure, which is preferably 20 to 40 mm in length.

The device has one or more radiopaque markers to provide visualization to the treating physician. For example, the radiopaque markers may be platinum, palladium, platinum-iridium, tantalum, gold, tungsten, or other types of radiopaque metals. In another example, radiopaque filaments may be used for visualization purpose and placed at various points on the device. The radiopaque marker/filament could be incorporated on the distal end of the stent structure, proximal end of the stent structure, and/or anywhere in between the distal and proximal end of the stent structure. The stent structure, in particular the struts/wires of the stent structure, may also be coated with a coating of a radiopaque material, for example a gold coating. The radiopaque coating could have a thickness of 1 to 6 μm, for example. The radiopaque coating also may only cover the importance areas of the stent structure only, i.e. places with tissue contact such as the effective length of the stent structure, or the cylindrical part of the stent structure. The stent structure could be incorporated with both radiopaque coating and one or more radiopaque markers.

In one embodiment, the stent structure could be manufactured with laser cutting technique, or other technique known to those skilled in the art from a tube. Such tube could have a wall thickness of 30 to 300 μm, preferably of 40 to 200 μm and most preferably of 40 to 120 μm. In another embodiment, the stent structure could be made of interwoven wires through braiding technique, or other technique to those skilled in the art. Such wire could have a thickness of 30 to 300 μm, preferably 40 to 200 μm and most preferably 40 to 120 μm. A catheter is used to deliver the stent structure into the vasculature. During delivery, the stent structure is compressed radially and placed inside the catheter. Such catheter could have an inner diameter of 0.4 to 0.9 mm.

The stent structure, in its radially expanded state, has a generally cylindrical shape in part or in whole with openings distributed on the outer cylindrical surface. In other words, the stent structure has a grid or mesh surface structure with a large number of openings between struts/wires on the outer cylindrical surface.

In one embodiment, the stent structure is permeable to the outside. In another embodiment, a stent structure could have one or more membranes on the circumference. The term “orifice” refers to the lattice or mesh structure, regardless of whether the orifice is isolated from the environment by a membrane, i.e., even an orifice covered by a membrane is referred to as an orifice. A membrane can be applied to the outside or inside of the mesh structure, if required. It is also possible to embed the grid and/or mesh structure in a membrane. The membranes may be made of a polymeric material such as polytetrafluoroethylene, polyesters, polyamides, polyurethanes, polyolefins or polysulfones. Polycarbonaturethanes (PCU) are particularly preferred.

The present invention relates to both a method for vasospasm treatment and to a corresponding device, as well as to the use of the device. The present invention can be used to treat acute vasospasm, but the use of prophylactic is also possible. The device, according to the present invention, can be used in particular in the neurovascular field, but it is also possible to use it in the cardiovascular or peripheral field. All statements made with regards to the device apply in the same way to the method and vice versa.

The present invention and the technical field are explained in more detail below, with reference to the figures. It should be noted that the figures show particularly preferred embodiments of the present invention, however the present invention is not limited to the embodiments shown. In particular, the present invention includes, to the extent that it is technically or methodologically useful, any combination of the technical or methodological features listed in the claims or described in the description are relevant to the invention.

1. A method of treating a vasospasm with a stent structure, the method comprising,
placing a stent structure inside a catheter, wherein the stent structure has a radially expanded state configured to be positioned against the inner wall of a blood vessel; a radially compressed state configured to be movable within the catheter; and a proximal end of the stent structure joining a delivery wire;
inserting the catheter into a treatment site inside the blood vessel;
deploying the stent structure by releasing the stent structure from the catheter, and allowing the stent structure transitioning from its radially compressed state into its radially expanded state and engaging the blood vessel; and
moving the stent structure in a longitudinal direction inside the blood vessel.

2. The method according to claim 1, characterized in that the stent structure is kept steady upon deploying inside the blood vessel for 1 to 10 min before moving in the longitudinal direction inside the blood vessel.

3. The method according to claim 1, characterized in that the stent structure is retracted back into the catheter after moving in the longitudinal direction inside the blood vessel, and is subsequently removed from the blood vessel within the catheter.

4. The method according to claim 1, characterized in that the movement of the stent structure inside the blood vessel is in the proximal direction.

5. The method according to claim 1, characterized in that the stent structure is composed of struts or wires, wherein upon deployment, edges of the struts or wires are configured to contact the blood vessel.

6. The method according to claim 5, characterized in that the struts or wires have at least partially a triangular-shaped, a wedge-shaped, a trapezoidal-shaped, a blade-shaped, a diamond-shaped, or a quadrilateral-shaped cross-section.

7. The method according to claim 6, characterized in that the edges of the struts or wires formed along vertexes the cross-sectional shape are at a most radially outward portion of the stent structure.

8. (canceled)

9. A device for the treatment of a vasospasm comprising,
a stent structure formed with a plurality of struts or wires, wherein the stent structure has a radially expanded state configured to be positioned against the inner wall of a blood vessel, and a radially compressed state configured to be movable within a catheter;
a delivery wire connecting a proximal end of the stent structure;
wherein the plurality of struts or wires comprise radially outward portions and radially inward portions, wherein the radially outward portions of the struts or wires are relatively narrower than radially inward portions of the struts or wires.

10. The device according to claim 9, characterized in that the struts or wires have at least partially a quadrilateral-shaped, a triangular shaped, a blade-shaped, a diamond-shaped cross-section, or a quadrilateral-shaped cross-section.

11. (canceled)

12. The device according to claim 9, characterized in that the stent structure has an effective working length, and wherein in the radially expanded state, the stent structure exerts a substantially constant radial outward force along the entire effective working length.

13. The device according to claim 12, characterized in that the struts or wires have a larger cross-section in a proximal section and a distal section of the effective working length of the stent structure than in a middle portion of the effective working length of the stent structure.

14. The device according to claim 12, characterized in that a density of the struts or wires is higher in a proximal and a distal sections of the effective working length of the stent structure than in a middle portion of the effective working length of the stent structure.

15. The device according to claim 10, characterized in that vertexes the cross-sectional shape of the struts or wire forms radially outermost edges of the stent structure.

16. The device according to claim 10, characterized in that corners of the cross-sectional shape of the struts or wires are at a most radially outward portion of the stent structure.
